(19) United States
(12) Patent Application Publication
(10) Pub. No.: US 2023/0310811 A1
(43) Pub. Date: Oct. 5, 2023

(54) AIRWAY DILATION BALLOON WITH HOLLOW CORE

(71) Applicant: Acclarent, Inc., Irvine, CA (US)

(72) Inventors: Ehsan Shameli, Irvine, CA (US); Fatemeh Akbarian, Rancho Palos Verdes, CA (US)

(21) Appl. No.: 18/207,349

(22) Filed: Jun. 8, 2023

Related U.S. Application Data
(62) Division of application No. 16/402,293, filed on May 3, 2019.
(60) Provisional application No. 62/687,853, filed on Jun. 21, 2018.

(51) Int. Cl. A61M 25/10; A61M 25/00; A61M 29/02

(52) U.S. Cl. CPC A61M 25/1002; A61M 25/0097; A61M 29/02; A61M 25/0102; A61M 2025/1061; A61M 2025/1097; A61M 2210/1035

(57) ABSTRACT

A system to dilate a stenotic region of an airway of a patient includes a stylet and a dilation catheter. The dilation catheter includes a catheter shaft and an inflatable balloon. The catheter shaft defines a shaft lumen and is configured to receive at least a portion of the stylet. The shaft lumen is axially aligned with a first longitudinal axis. The inflatable balloon is in fluid communication with an inflation lumen. The inflatable balloon is configured to transition between non-expanded and expanded configurations using the inflation lumen. The inflatable balloon has an outer perimeter configured to contact the stenotic region of the airway when in the expanded configuration. The inflatable balloon includes a pass-through lumen that is axially aligned with a second longitudinal axis that is laterally offset a distance from the first longitudinal axis. The pass-through lumen is disposed completely within the outer perimeter of the inflatable balloon.

### PRIORITY

This application is a divisional of U.S. Pat. App. No. 16/402,293, entitled “Airway Dilation Balloon with Hollow Core,” filed May 3, 2019, and published as U.S. Pub. No. 2019/0388656 on Dec. 26, 2019, which claims priority to U.S. Provisional Pat. App. No. 62/687,853, entitled “Airway Dilation Balloon with Hollow Core,” filed Jun. 21, 2018, the disclosure of which is incorporated by reference herein.

### BACKGROUND

Airway stenosis (or “airway narrowing”) is a medical condition that occurs when some portion of a patient’s airway becomes narrowed or constricted, thus making breathing difficult. A stenosis may occur in any part of the airway (e.g., larynx, trachea, bronchi or a combination). A stenosis may occur in children or adults and may be due to many different causes. In some instances, an airway stenosis is acquired, meaning the patient is not born with the condition. Airway stenosis may be the result of trauma caused by intubation when a tube is placed in the airway for ventilation/breathing assistance in a patient who cannot sufficiently breathe without assistance. Intubation for prolonged periods of time may traumatize the airway, causing the formation of scar tissue that forms the stenosis.

Sometimes the cause of stenosis is unknown, such as in idiopathic subglottic stenosis. Subglottic stenosis is one form of airway stenosis that occurs in the larynx, below the glottis (the area of the larynx around the vocal cords). The disorder can be either congenital or acquired and can affect both adults and children. To correct subglottic stenosis, the lumen of the cricoid area may be expanded to increase airflow during breathing. Over the years, surgical correction of subglottic stenosis has been performed using various techniques.

Therapies for treating airway stenosis range from endoscopic treatments, such as dilation and laser resection, to open procedures, such as laryngotracheal reconstruction. For example, a series of rigid dilators of increasing diameter may be advanced down the airway of the patient, gradually expanding the constriction but also applying unwanted shear forces to the airway. Balloon catheters may also be used to perform airway dilation. One of the benefits of balloon dilation over rigid dilation is the application of radial force versus shear force, which may reduce the risk of mucosal trauma. Also, depending on the balloon catheter used, more precise amounts of pressure may be applied to suitably dilate the stenotic region of the airway.

Airway dilations using balloon catheters may be performed using angioplasty balloon catheters and peripheral balloon catheters, which are designed for dilating narrowed blood vessels. However, these balloon catheters have several limitations when used for dilating an airway stenosis. First, since these balloons catheters are not specifically designed to be used in the airway, the dimensions may not be optimized for use within pediatric and/or adult airways. Second, balloon catheters are generally not sized to allow convenient visualization of airway balloon dilation using an endoscope (e.g., laryngoscope or bronchoscope), and in fact in some cases it is not possible to view the airway dilation procedure using an endoscope. Third, balloon catheters used for vascular procedures may be very long and highly flexible, making them difficult to advance into a constriction in the airway, which may lead to a tendency of the balloons of such catheters to slip out of the constriction when inflated.

It would be desirable to have an airway stenosis balloon dilation system that is designed to be used in an airway, rather than in a blood vessel or some other anatomical structure. Ideally, such a system would have dimensions configured for use in an airway, would allow for visualization of at least part of an airway dilation procedure and/or of the system during the procedure, and could be advanced into (and maintained within) an airway constriction more easily than currently available balloon catheters. Additionally, it may be desirable to provide easily controlled inflation/deflation of a balloon in dilation procedures, including procedures that will be performed only by a single operator.

An example of an airway stenosis balloon dilation system that is designed to be used in an airway is the INSPIRA AIR^{®} Balloon Dilation System by Acclarent, Inc. of Irvine, California. Further examples are described in U.S. Pat. No. 9,905,364, entitled “Device and Method for Dilating an Airway Stenosis,” issued Aug. 4, 2015, the disclosure of which is incorporated by reference herein; and U.S. Pat. No. 9,913,964, entitled “System and Method for Dilating an Airway Stenosis,” issued Mar. 13, 2018, the disclosure of which is incorporated by reference herein.

While a variety of balloon dilation devices have been made and used, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic side view of a first exemplary dilation catheter that includes a hub, a catheter shaft, and a first exemplary inflatable balloon;

FIG. 2 depicts a schematic side view of a stylet configured to be used together with the dilation catheter of FIG. 1;

FIG. 3 depicts an enlarged schematic cross-sectional view of the catheter shaft shown in FIG. 1, taken across section line 3-3 of FIG. 1;

FIG. 4A depicts an enlarged schematic sectional view of a first exemplary system that includes the dilation catheter of FIG. 1 and the stylet of FIG. 2, with the inflatable balloon in a non-expanded configuration and disposed adjacent a stenotic region of the airway of a patient;

FIG. 4B depicts the system of FIG. 4A, but with the inflatable balloon in an expanded configuration and in contact with the stenotic region of the airway;

FIG. 5 depicts a schematic side view of a second exemplary dilation catheter that includes a hub, a catheter shaft, and a second exemplary inflatable balloon;

FIG. 6 depicts an enlarged schematic perspective view of the inflatable balloon of FIG. 5 with a pass-through lumen extending therethrough;

FIG. 7 depicts an enlarged schematic cross-sectional view of the of the dilation catheter including the inflatable balloon and the catheter shaft of FIG. 5, taken across section line 7-7 of FIG. 5;

FIG. 8A depicts an enlarged schematic sectional view of a second exemplary system that includes the dilation catheter of FIG. 5 and the stylet of FIG. 2, with the inflatable balloon in a non-expanded configuration and disposed generally adjacent the stenotic region of the airway of the patient; and

FIG. 8B depicts the system of FIG. 4A, but with the inflatable balloon in an expanded configuration and in contact with the stenotic region of the airway.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Exemplary Dilation Catheter System

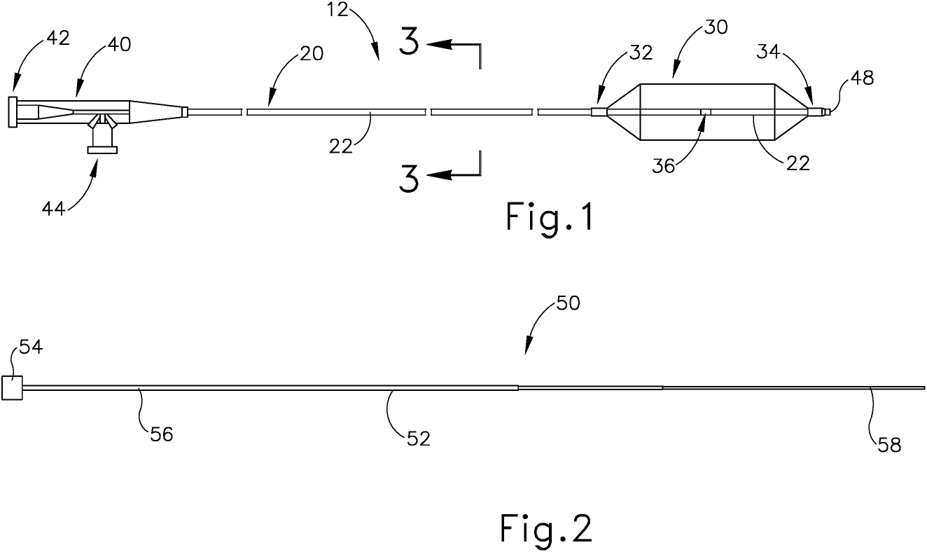

FIGS. 1-4B show a first exemplary system (10) that includes a first exemplary dilation catheter (12) and a first exemplary stylet (50) for dilating a stenotic region (S) in an airway (A) of a patient. As will be described in greater detail below, dilation catheter (12) and stylet (50) may be used together, with each having dimensions, stiffness characteristics, and other features specifically configured for dilation of airway (A).

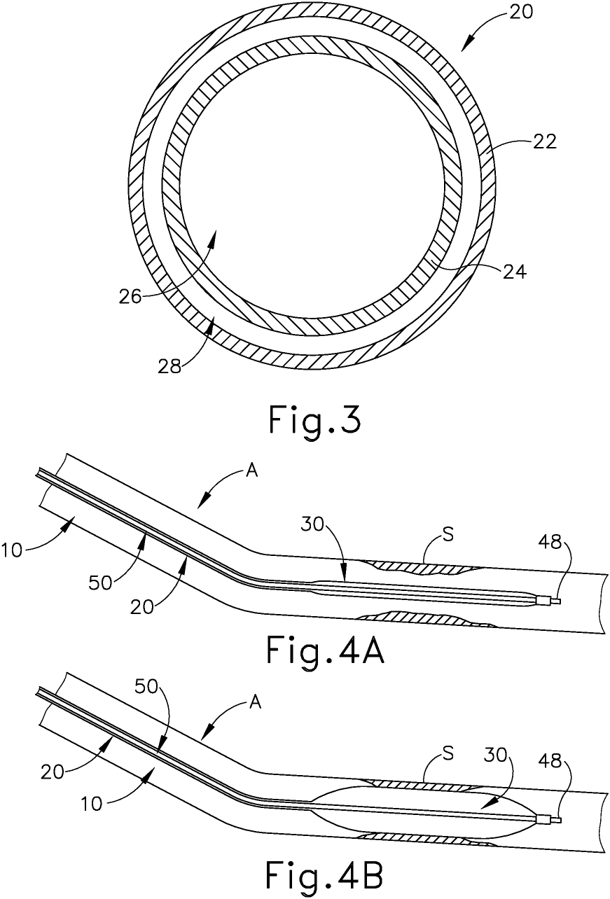

FIG. 1 shows dilation catheter (12) as including a catheter shaft (20), an inflatable balloon (30) and a hub (40). As shown, dilation catheter (12) includes a catheter shaft (20) having an outer tube (22) and an inner tube (24) (shown in FIG. 3). A distal tip (48) of catheter shaft (20) is shown as being enclosed to prevent stylet (50) from extending outside of distal tip (48). As shown in the cross-sectional view of FIG. 3, an inflation lumen (28) is formed between outer and inner tubes (22, 24). An inflation fluid (such as a saline or other solution) is intended to flow through an inflation port (44) of hub (40), into inflation lumen (28), and into inflatable balloon (30) to inflate inflatable balloon (30) from a non-expanded configuration shown in FIG. 4A to an expanded configuration shown in FIG. 4B. Catheter shaft (20), including outer tube (22) and inner tube (24), may be formed of any suitable material. One such suitable material is Pebax, although other materials may also be suitably utilized.

Outer and inner tubes (22, 24) may exhibit any suitable color and/or one or more markings. Any suitable combination, size, and color of markings may be used. For example, catheter shaft (20) may have a dark color, such as black or dark blue, and one or more light colored markings may be applied over the dark color. The markings (not shown) may include one or more of direct visualization markings or radiographic markings. Direct visualization markings may be viewed with the naked eye or with an endoscope to help the physician approximate the location of inflatable balloon (30) relative to certain anatomic features, such as a stenotic region (S). Radiographic markings may be viewed with a radiographic device such as intraoperative fluoroscopy to determine the position of inflatable balloon (30) relative to an airway constriction. For example, radiographic markings may be positioned on inner tube (24) and direct visualization markings may be positioned on outer tube (22).

FIG. 1 shows inflatable balloon (30) attached to catheter shaft (20) at a proximal attachment point (32) and a distal attachment point (34). Inflatable balloon (30) is in fluid communication with inflation lumen (28). As shown in the inflation sequence of FIGS. 4A and 4B described in greater detail below, dilation catheter (12) is inserted in airway (A) of the patient in the non-expanded configuration. After being positioned in the desired location, inflatable balloon (30) is inflated using inflation lumen (28) described above to dilate stenotic region (S) of airway (A) of the patient. A variety of balloon sizes and lengths may be provided, such that the physician may choose an appropriate size for an adult or pediatric patient. Any suitable material may be used for inflatable balloon (30). For example, inflatable balloon (30) may be made of nylon or another polymer, such as PTFE according to one particular example. Inflatable balloon (30) may include an outer slip-resistant surface, which may be formed by a textured surface or a coating.

FIG. 1 shows hub (40) as including a stylet port (42) and inflation port (44). Stylet port (42) is configured to provide a passageway for stylet (50) to be inserted into shaft lumen (26) of catheter shaft (20). Stylet port (42) is further configured to mate with a luer lock (54) of stylet (50) when stylet (50) is fully inserted in shaft lumen (26), so that dilation catheter (12) and stylet (50) are rotatably coupled, and together steered into a constricted portion of airway (A).

FIG. 2 shows stylet (50) in isolation. At least a portion of stylet (50) may have a greater stiffness than at least a portion of catheter shaft (20), so that when stylet (50) is bent and inserted within catheter shaft (20), catheter shaft (20) may at least partially conform to the shape of stylet (50). Stylet (50) includes a proximal portion (56) and a distal portion (58). Proximal portion (56) provides stiffness to dilation catheter (12) and enables dilation catheter (12) to be advanced through a patient’s nostril or mouth and into position within a stenotic region (S) of airway (A). By way of example only, catheter shaft (20) may have relatively weak column strength while stylet (50) has a relatively strong column strength, such that stylet (50) effectively imparts column strength to catheter shaft (20) when stylet (50) is coupled with dilation catheter (12).

Distal portion (58) of stylet (50) may include a bend or curve that is rigid enough to bend dilation catheter (12) during the placement of dilation catheter (12) within airway (A) of the patient. The bend may be pre-formed or formed on demand. Distal portion (58) may be malleable so that the user can selectively adjust the bend angle, and stylet (50) maintains the desired shape. This malleability allows a user to adjust a bend angle according to the airway anatomy of a particular patient. The bend may be maintained during and/or after when dilation catheter (12) is positioned in airway (A) of the patient. Stylet (50) may have a stiffness such that the bend partially or completely straightens out in airway (A) of the patient. This variation in flexibility along the length of stylet (50) may be achieved by using different materials, such as stainless steel and nitinol. These materials allow a physician to steer dilation catheter (12) using the bend. It is also envisioned that stylet (50) may be initially provided in a generally straight configuration. In some such versions, stylet (50) may be bent after it is fully seated in dilation catheter (12).

While not shown, stylet (50) may include a flexible atraumatic tip that extends distally out of catheter shaft (20) when stylet (50) is fully inserted within dilation catheter (12). This tip may facilitate the ability of a user to advance system (10) through airway (A) atraumatically. Additionally, while not shown, a coil may be disposed around at least part of distal portion (58) of stylet (50). However, stylet (50) may be used without a coil if desired. Stylet (50) may have an overall length approximately as long or slightly longer than catheter shaft (20) of dilation catheter (12). Additionally, the diameter of stylet (50) may be altered to achieve the variation in flexibility along the length of stylet (50). It is to be understood that stylet (50) may have any number of configurations and combinations of dimensions. As such, any of a number of different diameters, lengths, and the like may be used in forming stylet (50).

Stylet (50) may be permanently coupled with or removably coupled with dilation catheter (12). Dilation catheter (12) may include a locking mechanism (not shown) to lock stylet (50) in position within catheter shaft (20). The locking mechanism may include any suitable mechanical device, such as a lever, a ball and pin, and/or a luer lock feature. Alternatively, while not shown, stylet (50) may completely separate from dilation catheter (12), such that dilation catheter (12) and stylet (50) are configured to move independently. Use of stylet (50) while inserting dilation catheter (12) may help to guide the distal end of dilation catheter (12) through airway (A) of the patient and into stenotic region (S). Stylet (50) provides increased steerability and torquability during advancement of dilation catheter (12).

Dilation catheter (12) and/or stylet (50) may include illumination capability in the form of a light emitting portion, such as a light emitting distal end tip. For example, the distal tip of dilation catheter (12) and/or stylet (50) may be illuminated, such that the tip may be viewed from inside airway (A) using an endoscope and/or from outside the patient via transillumination. For example, dilation catheter (12) and/or stylet (50) may include one or more light fibers to transmit light from a light source attached to the proximal end of dilation catheter (12) and/or stylet (50) to its distal end. Light from a light emitting dilation catheter (12) and/or stylet (50) may be used to help a user visualize a patient’s airway (A).

FIGS. 4A and 4B show a method for dilating a stenotic region (S) of airway (A), (e.g. a subglottic stenosis) using system (10). More specifically, FIG. 4A shows inflatable balloon (30) of dilation catheter (12) in a non-expanded configuration during advancement and placement of dilation catheter (12). As described in detail above, system (10) may include dilation catheter (12) with inflatable balloon (30) disposed over stylet (50). System (10) may include an endoscope disposed adjacent to dilation catheter (12) for visualizing the placement of dilation catheter (12) in airway (A) of the patient. The method may include bending system (10) either by the user or manufacturer. For example, stylet (50) may be bent and subsequently inserted into dilation catheter (12). Alternatively, stylet (50) and dilation catheter (12) may be bent concurrently together, with stylet (50) already residing in dilation catheter (12). The support of stylet (50) and the bend may assist the physician to better navigate system (10) through airway (A) to position inflatable balloon (30) within at least a portion of stenotic region (S).

As shown in FIG. 4B, once the desired placement of system (10) has been achieved, and potentially verified using an endoscope, the method may include inflating inflatable balloon (30). FIG. 4B shows inflatable balloon (30) in the expanded configuration to dilate stenotic region (S). The physician inflates inflatable balloon (30) to a desired pressure. Inflatable balloon (30) may be inflated and deflated multiple times to sufficiently dilate stenotic region (S) of airway (A). The same or different pressures may be used in subsequent dilations. Moreover, proper dilation of stenotic region (S) may be confirmed by visualizing the region with the endoscope. The method also includes deflating inflatable balloon (30), and subsequently withdrawing dilation catheter (12) and stylet (50).

### II. Exemplary Alternative Dilation Catheter System With Ventilation Lumen Through Balloon

In the foregoing example, inflating inflatable balloon (30) of system (10) inside airway (A) completely blocks airway (A). This blockage, while temporary, may be undesirable. As a result, it may be more desirable to dilate the stenotic region (S) of airway (A) without completely blocking the airway (A) while balloon (30) is in an inflated state. This may allow balloon (30) to be inflated for a longer duration, without preventing the patient from being able to breathe while balloon (30) is inflated. By allowing balloon (30) to be inflated for a longer duration, the stenotic region (S) may be dilated more efficiently and/or effectively.

The following description provides additional details of an exemplary system (110) that includes an exemplary dilation catheter (112). Such system (110) and dilation catheter (112) described below may be used with any surgical instrument described above and below and in any of the various procedures described in the various patent references cited herein. As will be described in greater detailed below, system (110) and dilation catheter (112) may be used singularly or in combination with instruments, such as stylet (50). To this end, like numbers below indicate like features described above. Except as otherwise described below, system (110) described below may be constructed and operable like system (10) described above. Likewise, except as otherwise described below, dilation catheter (112) described below may be constructed and operable like dilation catheter (12) described above. Certain details of system (110) and dilation catheter (112) will therefore be omitted from the following description, it being understood that such details are already provided above in the description of system (10) and dilation catheter (12). Other suitable ways in which various surgical instruments may be used will be apparent to those of ordinary skill in the art in view of the teachings herein.

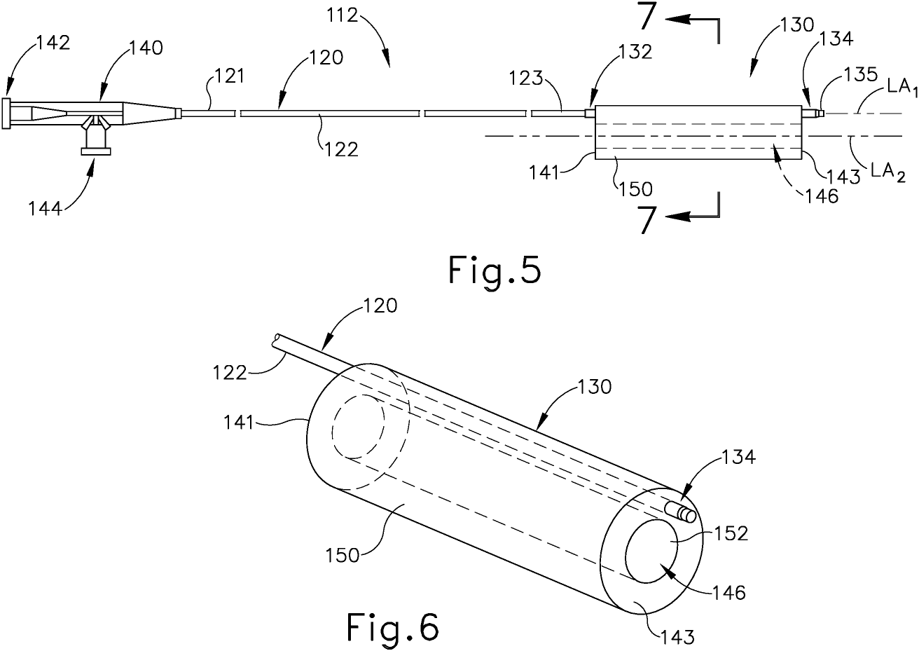

FIGS. 5-8B show second exemplary system (110) including second exemplary dilation catheter (112) and stylet (50) for dilating stenotic region (S) of airway (A) of the patient. As will be described in greater detail below, inflatable balloon (130) enables the patient to breathe naturally through a pass-through lumen (146) that is defined by inflatable balloon (130).

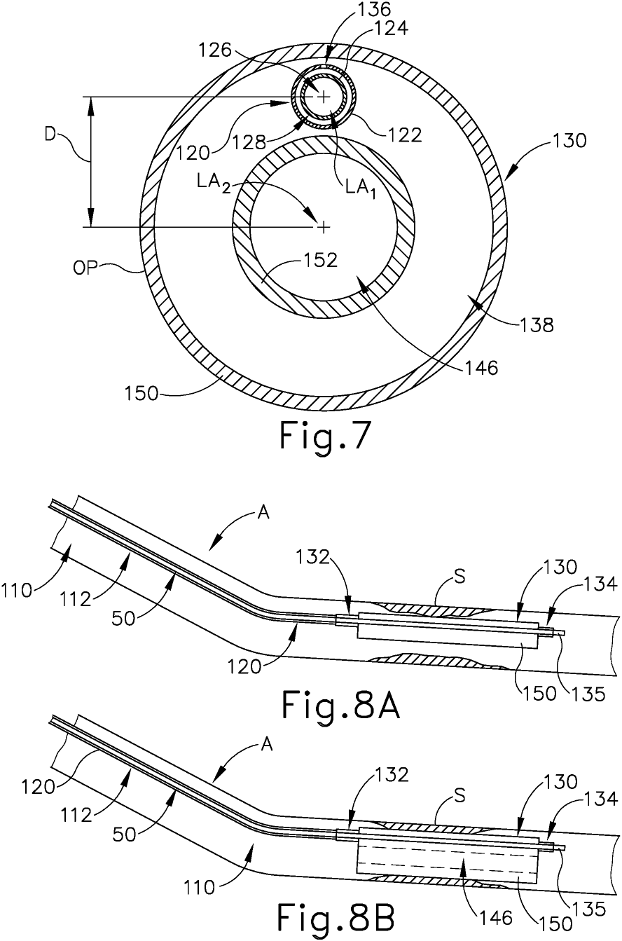

FIG. 5 shows dilation catheter (12) as including a catheter shaft (120), inflatable balloon (130), and a hub (140). Hub (140) includes a stylet port (142) and an inflation port (144). As shown, catheter shaft (120) includes a proximal portion (121) and a distal portion (123). Catheter shaft (120) includes an atraumatic distal tip (135) disposed adjacent distal portion (123). Catheter shaft (120) includes an outer tube (122) and an inner tube (124) (shown in FIG. 7) extending between proximal and distal portions (121, 123) of catheter shaft (110). As shown in FIG. 5, catheter shaft (110) is axially centered along a first longitudinal axis (LA₁). As shown in the cross-sectional view of FIG. 7, outer tube (122) concentrically surrounds inner tube (124) along first longitudinal axis (LA₁).

Inflatable balloon (130) is operatively coupled with distal portion (123) of catheter shaft (120). As shown in the cross-sectional view of FIG. 7, inflatable balloon (130) is in fluid communication with an inflation lumen (128) of catheter shaft (120) via a side port (136). Inflation lumen (128) extends radially and concentrically between outer and inner tubes (122, 124). As discussed below with respect to FIGS. 8A and 8B, inflatable balloon (130) is configured to transition from the non-expanded configuration shown in FIG. 8A to the expanded configuration shown in FIG. 8B by receiving inflation fluid from inflation lumen (128) via side port (136). Inflatable balloon (130) has an outer perimeter (OP) configured to contact stenotic region (S) of airway (A) when in the expanded configuration. Outer perimeter (OP) may contact the full circumference of airway (A) to dilate stenotic region (S).

Catheter shaft (120) defines a shaft lumen (126) configured to receive at least a portion of stylet (50) therethrough. Shaft lumen (126) extends along a first longitudinal axis (LA₁). More specifically, shaft lumen (126) extends radially within inner tube (124). As shown, inflatable balloon (130) does not expand concentrically around shaft lumen (126).

Inflatable balloon (130) includes an outer wall (150) and an inner wall (152). As shown in FIG. 7, outer wall (150) fully surrounds an inner wall (152) along the longitudinal direction of inflatable balloon (130), thereby defining radial boundaries of an inflation volume (138). Inflation volume (138) is in fluid communication with inflation lumen (128) of catheter shaft (120) via side port (136). Outer wall (150) defines outer perimeter (OP) of inflatable balloon (130). Inner wall (152) defines a pass-through lumen (146). As shown in FIGS. 6 and 7, catheter shaft (120) is contained between outer and inner walls (150, 152) of inflatable balloon (130). As shown in FIG. 6, inflatable balloon (130) generally forms a right circular hollow cylinder or a cylindrical shell, at least when in the expanded configuration. As shown in FIGS. 5 and 6, inflatable balloon (130) is coupled with catheter shaft (120) at proximal and distal attachment points (132, 134). Proximal attachment point (132) does not move relative to distal attachment point (134) when balloon (130) transitions from the non-expanded configuration to the expanded configuration. Alternatively, while not shown, proximal attachment point (132) may move relative to distal attachment point (134) when balloon (130) transitions from the non-expanded configuration to the expanded configuration.

As shown in FIG. 5, inflatable balloon (130) is axially centered along a second longitudinal axis (LA₂). As shown in the cross-sectional view of FIG. 7, inflation volume (138) of balloon (130) is also axially centered along second longitudinal axis (LA₂), which is laterally offset a distance (D) from first longitudinal axis (LA₁). Pass-through lumen (146) is also axially centered along second longitudinal axis (LA₂) and is configured to allow gas (such as air or oxygen) and/or fluid to pass therethrough while inflatable balloon (130) is in the expanded configuration. In the non-expanded configuration, pass-through lumen (146) may or may not be configured to allow gas/fluid to pass therethrough. As shown, pass-through lumen (146) is not concentric with shaft lumen (126). Pass-through lumen (146) is disposed completely within outer perimeter (OP) of inflatable balloon (130). As shown in FIG. 6, the cross-sectional area is generally the same from and between a proximal surface (141) of inflatable balloon (130) to a distal surface (143) of inflatable balloon (130), such that pass-through lumen (146) forms a generally cylindrical void or passageway defined by inner wall (152). However, it is envisioned that pass-through lumen (146) may have various shapes and/or sizes. While proximal and distal surfaces (141, 143) of inflatable balloon (130) are shown as generally planar, proximal and distal surfaces (141, 143) may be arcuate or angled if desired. It is also appreciated that pass-through lumen (146) may instead be two or more separate lumens if desired.

FIGS. 8A and 8B show an exemplary method for dilating stenotic region (S) in airway (A) of a patient. The method includes advancing a distal portion (123) of catheter shaft (120) through the mouth of the patient and into airway (A) of the patient. It is to be understood that dilation catheter (112) and stylet (50) may be advanced to stenotic region (S) of airway (A) together or separately. FIG. 8A shows inflatable balloon (130) in the non-expanded configuration and disposed adjacent the stenotic region (S). Once inflatable balloon (130) is properly positioned within stenotic region (S) of airway (A), inflatable balloon (130) may be inflated to the expanded configuration to dilate stenotic region (S) of airway (A).

Stylet (50) may remain within dilation catheter (112) during inflation of inflatable balloon (130). Maintaining stylet (50) within dilation catheter (112) during inflation may provide dilation catheter (112) with added column strength and help maintain the position of inflatable balloon (130) within stenotic region (S) of airway (A). Stylet (50) may be formed such that the bent or curved region of stylet (50) straightens out once dilation catheter (112) is positioned within airway (A). The bend may be retained even when positioned in airway (A) if desired. Alternatively, stylet (50) may be removed from dilation catheter (112) before inflating inflatable balloon (130). More specifically, stylet (50) may be removed from shaft lumen (126) of dilation catheter (112) after dilation catheter (112) is properly positioned within airway (A) of the patient and before inflation of inflatable balloon (130).

FIG. 8B shows inflatable balloon (130) in the expanded configuration, such that outer perimeter (OP) of inflatable balloon (130) is in contact with stenotic region (S) of airway (A). With inflatable balloon (130) in the inflated configuration, pass-through lumen (146) allows air to through the entire length of balloon (130), such that the flow of air through airway (A) is not completely blocked by inflated balloon (130). As shown in FIG. 6, volume (138) and pass-through lumen (146) are disposed completely within outer perimeter (OP) of inflatable balloon (130) and are not concentric with shaft lumen (126) defined by catheter shaft (120). Instead, volume (138) and pass-through lumen (146) extend along second longitudinal axis (LA₂), which is laterally offset by distance (D) from first longitudinal axis (LA₁). As a result, air may pass through pass-through lumen (146) that is defined by inner wall (152). It is envisioned that the inflating and deflating steps may be performed repeatedly (e.g. two or more times) if desired. This repeated inflation be at the same or different pressures. Due to the presence of pass-through lumen (146) it may be possible to leave inflatable balloon (130) in an inflated state in stenotic region (S) for a duration longer than that permitted by balloon (30) of dilation catheter (12).

Similar to system (110) as shown in FIG. 8A, after inflatable balloon (130) is inflated and the constriction of airway (A) corrected, the method also includes deflating inflatable balloon (130), so that outer perimeter (OP) of inflatable balloon (130) does not contact stenotic region (S) of airway (A). After deflating inflatable balloon (130), the method also includes withdrawing dilation catheter (112) from stenotic region (S) of airway (A). It is appreciated that dilation catheter (112) and stylet (50) may be withdrawn from stenotic region (S) of airway (A) together or separately. For example, stylet (50) may be removed after the stenosis has been dilated but before dilation catheter (112) is removed from the patient.

The method may also include advancing an endoscope (not shown) along airway (A) of the patient and positioning a distal end of the endoscope near stenotic region (S) to visualize placement of system (110) including inflatable balloon (130) prior to, during, or after inflation of inflatable balloon (130). For example, if desired, the endoscope may be attached to dilation catheter (112) using a coupling member, to help prevent movement and slippage during dilation of inflatable balloon (130). After the dilation is performed, the endoscope may be detached from the grip and removed from the patient. Alternatively, the endoscope may be separate from dilation catheter (112). The endoscope may be positioned alongside dilation catheter (112) or the endoscope may be positioned within or through dilation catheter (112). The method of dilating the stenosis may include inserting an endoscope into airway (A) of the patient and then passing dilation catheter (112) through the endoscope.

The system (110) and method described above increase the ease of use for the physician performing the dilation of stenotic region (S) of airway (A) of the patient. The physician may manipulate system (110) using one hand, thus leaving the other hand free to hold the endoscope or other device. The combination of dilation catheter (112), with its advantageous length, shaft and balloon diameters and overall configuration, and stylet (50), with its bend to facilitate airway navigation, may make an airway dilation procedure easier and more often successful. Additionally, the atraumatic design of dilation catheter (112) and stylet (50) may help prevent damage to airway (A) and vocal cords of the patient during delivery and removal. Additionally, system (110) may help prevent movement and slippage of dilation catheter (112) during dilation of stenotic region (S), which may in turn provide a more controlled dilation. Moreover, inflatable balloon (130) may significantly reduce risks associated with an airway dilation procedure by enabling the patient to breathe substantially naturally through pass-through lumen (146) disposed within inflatable balloon (130) while balloon (130) is in the inflated state.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system for dilating a stenotic region of an airway of a patient, the system comprising: (a) a stylet; and (b) a dilation catheter comprising: (i) a catheter shaft having proximal and distal portions, wherein the catheter shaft defines: (A) a shaft lumen configured to receive at least a portion of the stylet therethrough, wherein the shaft lumen is axially aligned with a first longitudinal axis, and (B) an inflation lumen, and (ii) an inflatable balloon operatively coupled with the distal portion of the catheter shaft and in fluid communication with the inflation lumen of the catheter shaft, wherein the inflatable balloon is configured to transition between non-expanded and expanded configurations using the inflation lumen, and wherein the inflatable balloon has an outer perimeter configured to contact the stenotic region of the airway when in the expanded configuration, wherein the inflatable balloon includes a pass-through lumen that is axially aligned with a second longitudinal axis that is laterally offset a distance from the first longitudinal axis, and wherein the pass-through lumen is disposed completely within the outer perimeter of the inflatable balloon.

### Example 2

The system of Example 1, wherein the inflatable balloon is configured to not expand concentrically around the shaft lumen.

### Example 3

The system of any one or more of Examples 1 through 2, wherein the inflatable balloon includes inner and outer walls, wherein the outer wall defines the outer perimeter, and wherein the inner wall defines the pass-through lumen that is configured to enable air to pass therethrough while the inflatable balloon is in the expanded configuration.

### Example 4

The system of any one or more of Examples 1 through 3, wherein the outer wall surrounds the inner wall along the second longitudinal axis.

### Example 5

The system of any one or more of Examples 1 through 4, wherein the outer wall concentrically surrounds the inner wall along the second longitudinal axis.

### Example 6

The system of any one or more of Examples 1 through 5, wherein at least a portion of the catheter shaft is contained between the inner and outer walls of the inflatable balloon.

### Example 7

The system of any one or more of Examples 1 through 6, wherein the inflatable balloon generally forms a hollow cylinder shape at least when in the expanded configuration

### Example 8

The system of any one or more of Examples 1 through 7, wherein the catheter shaft includes an outer tube concentrically surrounding an inner tube, wherein the inflation lumen extends radially between the inner and outer tubes, and wherein the shaft lumen extends radially within the inner tube and is configured to receive the stylet.

### Example 9

The system of any one or more of Examples 1 through 8, wherein the proximal portion of the catheter shaft further comprises a hub, wherein a proximal portion of the stylet includes a luer lock configured to couple with the hub.

### Example 10

The system of any one or more of Examples 1 through 9, wherein the catheter shaft further includes an inner tube and an outer tube extending between the proximal and distal portions of the shaft, wherein the outer tube concentrically surrounds the inner tube along the first longitudinal axis.

### Example 11

The system of any one or more of Examples 1 through 10, wherein the inner tube includes a side port, and wherein the inflatable balloon is configured to transition from the non-expanded configuration to the expanded configuration by receiving inflation fluid through the side port from the inflation lumen.

### Example 12

The system of any one or more of Examples 1 through 11, wherein the inflatable balloon is coupled with the catheter shaft at proximal and distal attachment points, wherein the proximal attachment point is configured to not move relative to the distal attachment point when transitioning from the non-expanded configuration to the expanded configuration.

### Example 13

The system of any one or more of Examples 1 through 12, wherein the stylet is malleable.

### Example 14

The system of any one or more of Examples 1 through 13, wherein the catheter shaft includes an atraumatic distal tip.

### Example 15

A system for dilating a stenotic region of an airway of a patient, the system comprising: (a) a stylet; and (b) a dilation catheter comprising: (i) a catheter shaft having proximal and distal portions, wherein the catheter shaft defines: (A) a shaft lumen configured to receive at least a portion of the stylet therethrough, and (B) an inflation lumen; and (ii) an inflatable balloon operatively coupled with the distal portion of the catheter shaft, wherein the inflatable balloon defines an inflation volume in fluid communication with the inflation lumen of the catheter shaft, wherein the inflatable balloon is configured to transition between non-expanded and expanded configurations using the inflation lumen, wherein the inflatable balloon has an outer perimeter configured to contact the stenotic region of the airway when in the expanded configuration, wherein the inflatable balloon includes a pass-through lumen this is not concentric with the shaft lumen, wherein the pass-through lumen is in fluid isolation relative to the inflation volume, and wherein the pass-through lumen is disposed completely within the outer perimeter of the inflatable balloon.

### Example 16

The system of any one or more of Examples 1 through 15, wherein the inflatable balloon includes inner and outer walls, wherein the inner and outer walls cooperate to define the inflation volume, wherein the outer wall further defines the outer perimeter, and wherein the inner wall further defines the pass-through lumen that is configured to enable air to pass therethrough while the inflatable balloon is in the expanded configuration.

### Example 17

A method for dilating a stenotic region in an airway of a patient, the method comprising: (a) advancing a distal portion of a catheter shaft of a dilation catheter through the mouth of the patient and into the airway of the patient, wherein the dilation catheter includes an inflatable balloon operatively coupled with the distal portion of the catheter shaft, wherein the inflatable balloon is configured to transition between non-expanded and expanded configurations, wherein the inflatable balloon reaches the stenotic region in the non-expanded configuration; (b) inflating the inflatable balloon from the non-expanded configuration to the expanded configuration so that an outer perimeter of the inflatable balloon contacts the stenotic region of the airway, wherein the inflatable balloon includes a pass-through lumen that allows air to pass therethrough while the inflatable balloon is in the expanded configuration, wherein the pass-through lumen is disposed completely within the outer perimeter of the inflatable balloon and is not concentric with a shaft lumen defined by the catheter shaft; (c) deflating the inflatable balloon so that the outer perimeter of the inflatable balloon does not contact the stenotic region of the airway; and (d) withdrawing the dilation catheter from the stenotic region of the airway.

### Example 18

The method of Example 17, wherein the inflatable balloon includes inner and outer walls, wherein the outer wall defines the outer perimeter, and wherein the inner wall defines the pass-through lumen that allows air to pass therethrough while the inflatable balloon is in the expanded configuration.

### Example 19

The method of any one or more of Examples 17 through 18, wherein steps (b) and (c) are performed at least twice.

### Example 20

The method of any one or more of Examples 17 through 19, wherein advancing the distal portion of the catheter shaft of the dilation catheter further includes advancing the distal portion of the catheter shaft of the dilation catheter with a stylet disposed at least partially within the shaft lumen, and wherein withdrawing the dilation catheter further comprises withdrawing the dilation catheter and the stylet from the stenotic region of the airway together or separately.

### IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1-16. (canceled)

17. A method for dilating a stenotic region in an airway of a patient, the method comprising:
(a) advancing a distal portion of a catheter shaft of a dilation catheter through the mouth of the patient and into the airway of the patient, the dilation catheter including an inflatable balloon operatively coupled with the distal portion of the catheter shaft, the inflatable balloon being configured to transition between non-expanded and expanded configurations, the inflatable balloon reaching the stenotic region in the non-expanded configuration;
(b) inflating the inflatable balloon from the non-expanded configuration to the expanded configuration so that an outer perimeter of the inflatable balloon contacts the stenotic region of the airway, the inflatable balloon including a pass-through lumen that allows air to pass therethrough while the inflatable balloon is in the expanded configuration, the pass-through lumen being disposed completely within the outer perimeter of the inflatable balloon and being not concentric with a shaft lumen defined by the catheter shaft;
(c) deflating the inflatable balloon so that the outer perimeter of the inflatable balloon does not contact the stenotic region of the airway; and
(d) withdrawing the dilation catheter from the stenotic region of the airway.

18. The method of claim 17, the inflatable balloon including inner and outer walls, the outer wall defining the outer perimeter, and the inner wall defining the pass-through lumen that allows air to pass therethrough while the inflatable balloon is in the expanded configuration.

19. The method of claim 17, steps (b) and (c) being performed at least twice.

20. The method of claim 17, advancing the distal portion of the catheter shaft of the dilation catheter further including advancing the distal portion of the catheter shaft of the dilation catheter with a stylet disposed at least partially within the shaft lumen, and withdrawing the dilation catheter further comprising withdrawing the dilation catheter and the stylet from the stenotic region of the airway together or separately.

21. The method of claim 20, the stylet having a first column strength, the catheter shaft having a second column strength less than the first column strength of the stylet.

22. The method of claim 20, the catheter shaft defining a stop element configured to prevent the stylet from extending distally beyond the stop element.

23. The method of claim 22, the stop element including a completely enclosed atraumatic distal tip.

24. The method of claim 20, a distal portion of the stylet including a preformed bend or a malleable bend, the preformed bend or the malleable bend being configured to be sufficiently rigid to bend the dilation catheter during the act of advancing the distal portion of the catheter shaft of the dilation catheter.

25. The method of claim 17, the airway including a larynx.

26. The method of claim 25, the patient including an adult patient.

27. The method of claim 17, the airway including a trachea.

28. The method of claim 27, the patient including an adult patient.

29. The method of claim 17, inflating the inflatable balloon including dilating the stenotic region of the airway.

30. A method for dilating a stenotic region in an airway of a patient, the method comprising:
(a) advancing a distal portion of a catheter shaft of a dilation catheter into the airway of the patient while an inflatable balloon of the dilation catheter is in a non-expanded configuration, the catheter shaft extending along a first longitudinal axis;
(b) arresting advancement of the distal portion of the catheter shaft when the inflatable balloon of the dilation catheter reaches the stenotic region of the airway;
(c) inflating the inflatable balloon from the non-expanded configuration to an expanded configuration so that an outer perimeter of the inflatable balloon engages the stenotic region of the airway and thereby dilates the stenotic region of the airway;
(d) allowing air within the airway to pass through a pass-through lumen of the inflatable balloon while the inflatable balloon is in the expanded configuration, the pass-through lumen being disposed completely within the outer perimeter of the inflatable balloon and extending along a second longitudinal axis that is laterally offset from the first longitudinal axis;
(e) deflating the inflatable balloon so that the outer perimeter of the inflatable balloon disengages the stenotic region of the airway; and
(f) withdrawing the dilation catheter from the stenotic region of the airway.

31. The method of claim 30, advancing the distal portion of the catheter shaft including advancing the distal portion of the catheter shaft through the mouth of the patient.

32. The method of claim 30, the airway including at least one of a larynx or a trachea.

33. The method of claim 32, the patient including an adult patient.

34. A method for dilating a stenotic region in an airway of a patient, the method comprising:
(a) advancing a distal portion of a catheter shaft of a dilation catheter into the airway of the patient while an inflatable balloon of the dilation catheter is in a non-expanded configuration, the catheter shaft extending along a first longitudinal axis;
(b) arresting advancement of the distal portion of the catheter shaft when the inflatable balloon of the dilation catheter reaches the stenotic region of the airway;
(c) communicating an inflation fluid through an inflation lumen of the catheter shaft to an inflation volume of the inflatable balloon to inflate the inflatable balloon from the non-expanded configuration to an expanded configuration so that an outer perimeter of the inflatable balloon engages the stenotic region of the airway and thereby dilates the stenotic region of the airway;
(d) allowing air within the airway to pass through a pass-through lumen of the inflatable balloon while the inflatable balloon is in the expanded configuration, the pass-through lumen being in fluid isolation relative to the inflation volume and extending along a second longitudinal axis that is laterally offset from the first longitudinal axis;
(e) deflating the inflatable balloon so that the outer perimeter of the inflatable balloon disengages the stenotic region of the airway; and
(f) withdrawing the dilation catheter from the stenotic region of the airway.

35. The method of claim 34, the airway including at least one of a larynx or a trachea.

36. The method of claim 35, the patient including an adult patient.
